(19) United States
(12) Patent Application Publication
(10) Pub. No.: US 2010/0324583 A1
(43) Pub. Date: Dec. 23, 2010

(54) Surgical Instrument

(75) Inventor: Reiner Haag, Spaichingen (DE)

Correspondence Address: ALLEMAN HALL MCCOY RUSSELL & TUTTLE LLP, 806 SW BROADWAY, SUITE 600, PORTLAND, OR, 97205-3335, US

(73) Assignee: LAWTON GMBH & CO. KG, Fridingen (DE)

(21) Appl. No.: 12/815,187

(22) Filed: Jun. 14, 2010

(30) Foreign Application Priority Data
Jun. 17, 2009 (DE) 20 2009008 370.5U

(51) Int. Cl. A61B 17/34

(52) U.S. Cl. 606/185

(57) ABSTRACT

The present disclosure relates to a surgical instrument with a main part with a first punching means and a part movable relative thereto, with a handle arranged on the main part, which includes a stationary grip and a grip which can actuate the movable part, wherein the movable part is releasably connected with a second punching means, and wherein the second punching means can be joined with the first punching means for a punching operation and along with the movable part is movably guidable or guided on the main part.

### CROSS-REFERENCE TO RELATED APPLICATION

This application claims priority to German Utility Model Application No. 20 2008 008 370.5, filed Jun. 17, 2009, entitled “Surgical Instrument”, which is hereby incorporated by reference in its entirety for all purposes.

### TECHNICAL FIELD

The present disclosure relates to a surgical instrument with a main part with a first punching means and a part movable relative thereto, with a handle arranged on the main part, which includes a stationary grip and a grip which can actuate the movable part.

Such surgical instruments are used for example for laminectomies or hemilaminectomies. These are surgical procedures, in which parts of bones and/or cartilages are punched out.

DE 197 42 112 A1 relates to a surgical sliding shaft instrument, comprising a shaft portion and grip and finger branches as well as a slide longitudinally movable against a spring force by means of the finger branch, which slide is longitudinally movably guided on the shaft and can be actuated by means of the finger branch. By loosening a screw connection, the instrument can be dismantled into all its parts by means of a handle, without leaving parts connected with each other in any way.

DE 199 49 422 A1 describes a surgical instrument with a main part and at least one part movable relative thereto, with a handle arranged on the main part, which includes a stationary grip and a grip which can actuate the movable part, wherein a locking means is provided, by means of which the movable part can captively be swiveled away from the main part.

Such surgical instruments also are referred to as bone punches, since parts of bones and/or cartilages can be punched out by means of such instruments. One field of application is laminectomy or hemilaminectomy.

During the laminectomy, the vertebral arch is removed along with the spinous process of one or more vertebrae, in order to gain space for a spinal-cord or intervertebral-disk surgery or to be able to relieve an excessive pressure, for example due to a tumor in the vertebral canal. Since this surgery can lead to phenomena of instability in the region of the spine, in particular to luxations, a hemilaminectomy is performed quite frequently. There is merely removed one half of the vertebral arch.

Thus, a considerable amount of bone or cartilage must be removed in the course of the surgical procedure. This inevitably leads to phenomena of wear at the cutting edge of the punch instrument, i.e. the punch gradually loses its sharpness and becomes blunt. During the operation, this will among other things lead to the fact that the attending physician or surgeon tries to nevertheless remove the tissue, even partly tear off the same, by rotating or ripping movements. Consequently, deformations may possibly occur at the punch and/or the tissue is not removed neatly and in a defined way, whereby the healing process can be extended.

Working with blunt instruments in the surgical procedures described above also can have disadvantageous consequences for the operating surgeon. Due to the great effort, e.g. due to the rotating and ripping movements, the operating surgeon can develop tendovaginitis and/or joint disorders, since in general several operations must be performed one after the other on an operation day.

Therefore, it is the object of the present disclosure to develop a surgical instrument as mentioned above in an advantageous way, in particular to the effect that the same provides for a safe and easy handling while ensuring good sterilizability, preferably that a sufficient sharpness of the punch can be ensured at reasonable costs.

In accordance with the present disclosure, this object is solved by a surgical instrument. Accordingly, it is provided that a surgical instrument with a main part is provided with a first punching means and a part movable relative thereto and with a handle arranged on the main part, which includes a stationary grip and a grip which can actuate the movable part. It is furthermore provided that the movable part is releasably connected with a second punching means, wherein the second punching means can be joined with the first punching means for a punching operation and is movably guidable or guided on the main part along with the movable part.

Such surgical instrument for example can be a so-called bone punch for a laminectomy or a hemilaminectomy. The main part and the first punching means can be formed in one piece, but it can just as well also be provided that the first punching means is releasably attached to the main part. The first and second punching means each preferably include a tray-like formation, such as a punch tray with sharp tray ends, so that by joining the first and second punching means tissue such as bone or cartilage can be punched out by the tray ends of the punch tray. The two punch trays preferably form a space in the closed or joined condition of the two punching means, in which the tissue material punched out is located. Due to the fact that the second punching means and the movable part are releasably connected with each other, it is possible to configure the second punching means subject to wear in use as a replacement part. This provides for simply replacing the second punching means when a corresponding degree of wear thereof is reached. For example, it can be provided to configure the second punching means as a disposable part.

Furthermore, it is possible that both the second punching means and the movable part include guiding means, by means of which the second punching means and the movable part are movably guidable or guided in a guideway in the main part. This involves the advantage to provide for a defined relative movement of the second punching means and the movable part relative to the main part.

It can furthermore be provided that the second punching means and the movable part are positively guidable or guided on or in the main part.

Moreover, it is conceivable that the main part includes an at least partly formed groove in which the second punching means and the movable part are at least partly movably guidable or guided on or in the main part. In this connection it is conceivable for example that the guiding means of the second punching means and of the movable part only partly engage in the groove in a first position and completely engage in the groove in a second position. It can also be provided that in a first position only the guiding means of the second punching means engage in the groove and in a second position the guiding means both of the second punching means and of the movable part engage in the groove. The first position can be an open position and the second position can be a closed position of the surgical instrument.

Particularly advantageously, the groove is dovetailed.

In addition, it can be provided that the guiding means of the second punching means and of the movable part are at least partly dovetailed and at least partly positively engage in the groove. For example, the guiding means can be formed by dovetail tongues which are provided for engagement in a corresponding groove of the main part.

Moreover, it is conceivable that the movable part is positively releasably connected with the second punching means.

It is furthermore possible that the positively releasable connection between the movable part and the second punching means is at least partly dovetailed.

Thus, it can be particularly advantageous that the second punching means is connected with the main part and the movable part in a doubly dovetailed manner, wherein the connection with the main part consists in a movable guidance with the dovetailed groove of the main part. Due to this clearly specified positive connection, the user only is given one single possibility of how to insert the second punching means into the surgical instrument. The handleability of the surgical instrument thereby is increased in general.

The second punching means for example can only be a small punch tip, but also a longer punch shaft, which can extend over an essential part of the length of the main part of the surgical instrument.

Furthermore, it is conceivable that the second punching means is a hardened material, in particular a hardened surgical steel.

Particularly advantageously, the hardness of the second punching means or of the first punching means is at least 45 HRC, preferably 52 HRC, particularly preferably 56-60 HRC (known as Rockwell Hardness). In this connection it is conceivable for example that the first punching means is a separately hardened point or a separately hardened region of the main part of the surgical instrument.

It is furthermore particularly advantageous when the surgical instrument can be dismantled into all its parts. This provides a particularly advantageous sterilizability, since each individual part can be cleaned well initially and subsequently can be sterilized from all sides.

It can be provided that at least one unlocking means is present, by means of which main part and movable part can be unlocked or detached from the handle.

It is particularly advantageous when main part, movable part and second punching means substantially can be separated from the handle in one single operation.

Further details and advantages of the present disclosure will now be explained in detail with reference to an embodiment illustrated in the drawings.

### BRIEF DESCRIPTION OF THE FIGURES

FIG. 1 shows views of a surgical instrument in an open position.

FIG. 2 shows views of a surgical instrument in a closed position.

FIG. 3 shows views of the main part of the surgical instrument.

FIG. 4 shows views of the movable part of the surgical instrument.

FIG. 5 shows views of the second punching means. The figures are drawn approximately to scale with respect to one another, with the detail views drawn to a larger scale.

### DETAILED DESCRIPTION

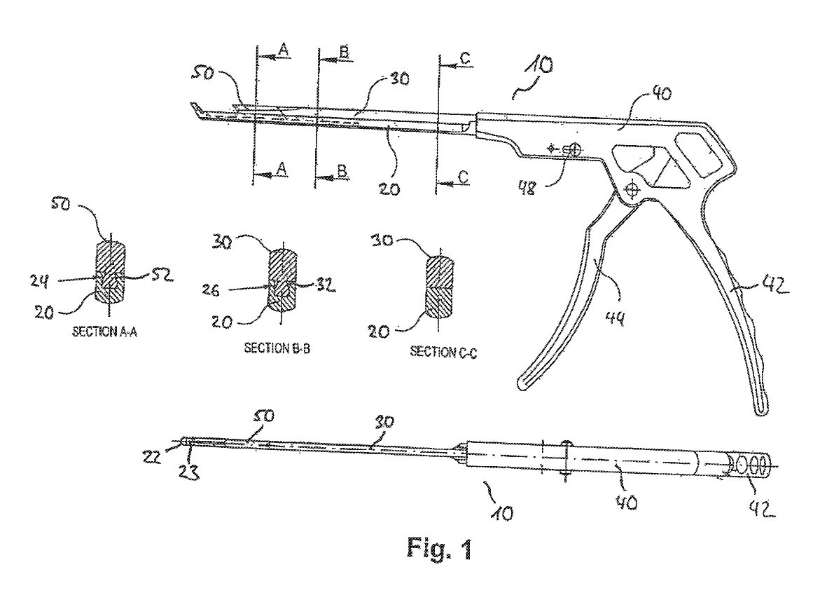

FIG. 1 shows several views of a surgical instrument 10 of the present disclosure in an open position. The open position of the surgical instrument 10 is the position in which the first and the second punching means 22, 50 are not joined.

The surgical instrument includes a handle 40 in which the main part 20 is engaged. Like the main part 20, the movable part 30 which is guided on the main part 20 is engaged in the handle 40. As shown, the handle may be sized and shaped to be squeezed by a human hand.

By means of the unlocking pin 48, main part 20 and movable part 30 can be unlocked and thus be separated from the handle 40.

The handle 40 includes a stationary grip 42 and a grip 44 to be actuated in the manner of a traction lever, by means of which the movable part 30 can be moved relative to the main part 20.

At the front end of the movable part 30 a second punching means 50 in the form of a replaceable punch tip 50 is arranged, which like the movable part 30 is movably guided into the main part 20. By actuating the movable grip 44, the movable part 30 and the second punching means 50 are joined with the first punching means 22.

The first punching means 22, which may include a punch, is integrally formed at the main part 20 as an L-shaped upstanding formation and includes a tray-like recess 23 which can also be referred to as punch tray 23. The tray ends of the punch tray 23 are sharp-edged, so that tissue material can be cut off therewith. The second punching means 50, which may also include a punch, forms a counterpart thereto, which will subsequently be described in detail.

The movable guidance of the second punching means 50 and of the movable part 30 on or in the main part 20 can be taken from the sections A-A, B-B and C-C. As can be taken from section A-A, the second punching means 50 with its dovetailed guiding means 52, which be a guide tongue, positively engages in a dovetailed groove 24 in the front region of the main part 20 and thus is positively movably guided in the main part 20. As can be taken from section B-B, the movable part 30 likewise includes a dovetailed guiding means 32, which partly engages in the dovetailed groove 24 of the main part 20, but otherwise is guided in a groove 26 with vertical walls. As can be taken from section C-C, main part 20 and movable part 30 lie on top of or on each other with smooth surfaces.

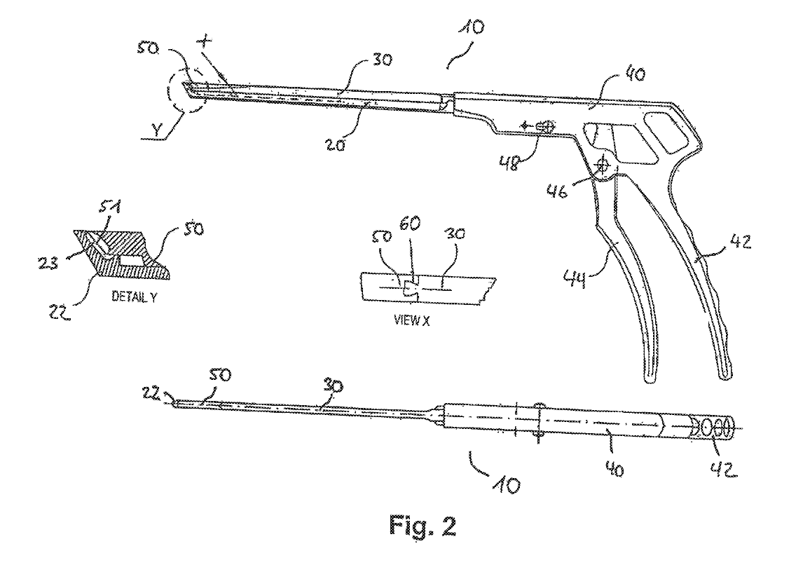

FIG. 2 shows the surgical instrument 10 in a closed position, i.e. the first and second punching means 22, 50 are joined. The closed condition of the surgical instrument 10 can be achieved in that the actuatable grip 44 is pulled. The actuable grip 44, which is rotatably mounted in the handle 40 at the pivot point 46, then pushes the movable part 30 and the second punching means 50 arranged at the front of the movable part 30 forwards against the first punching means 22.

The second punching means 50 also includes a punch tray 51, which has sharp-edged tray ends. In the joined condition of the first and second punching means 22, 50 a space is formed by the punch trays 23, 51, in which the tissue material punched out can be retained and thus safely be removed from the site of operation. This is shown in detail for example in the detail Y.

The movable part 30 and the second punching means 50 are connected with each other by means of a positive, dovetailed connection 60. The second punching means 50 and the movable part 30 thus can be joined and also be detached again in a safe and simple and unambiguous manner. This is shown in detail for example in the view X.

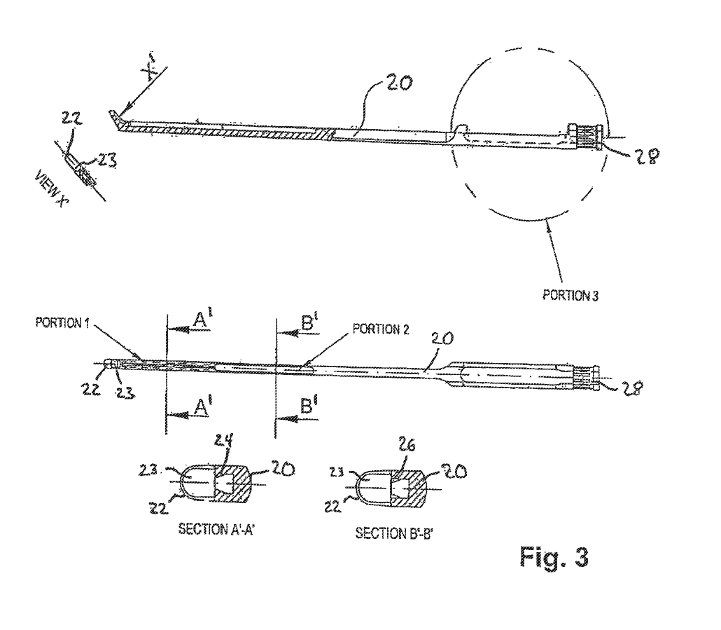

In FIG. 3, the main part shown in FIGS. 1 and 2 is illustrated in detail. The view X′ shows a direct view of the first punching means 22 with the punch tray 23. The main part includes two portions 1 and 2, wherein portion 1 is arranged in the front region adjacent to the first punching means 22. Portion 2 is arranged directly after portion 1. As can be taken from section N-A′, portion 1 includes the dovetailed groove 24 which extends along the entire length of portion 1. As can be taken from section B′-B′, portion 2 includes the groove 26 with the vertical side walls, wherein groove 26 extends along the entire length of portion 2.

At the rear end of the main part 20, the main part 20 includes a connecting means 28, by means of which the main part 20 can safely be mounted in the handle 40 in a precise position.

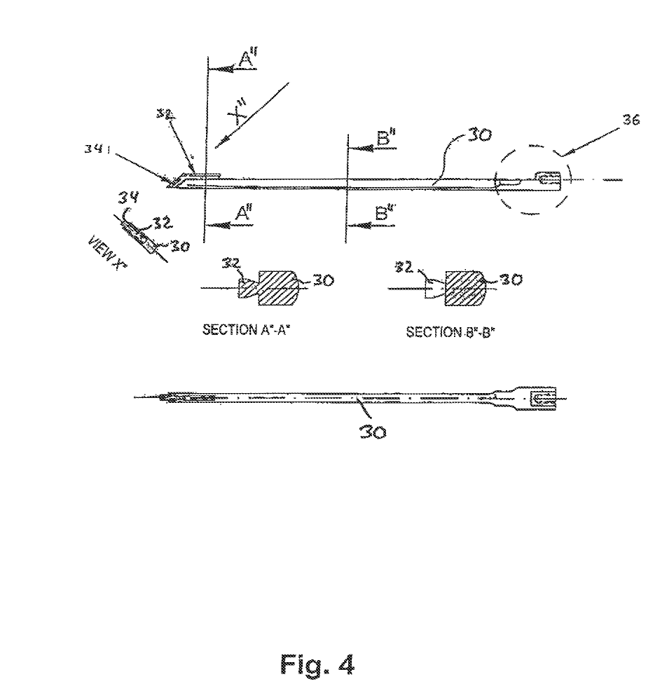

FIG. 4 shows several views and details of the movable part 30 of the surgical instrument 10, wherein the movable part 30 is shown in FIG. 4 in a position rotated by 180° as compared to FIGS. 1 and 2. As can be taken for example from view X″, the dovetailed formations intersect each other at the front end of the movable part 30 such that both insertion into the groove 24 and insertion into the dovetail groove of the second punching means 50 is possible.

The sections A″-A″ and B″-B″ each show sections through the movable part 30 at the points indicated in FIG. 4.

The movable part 30 also includes a connecting means 36 in its rear region, by means of which the movable part 30 can directly or indirectly be attached to the movable grip 44.

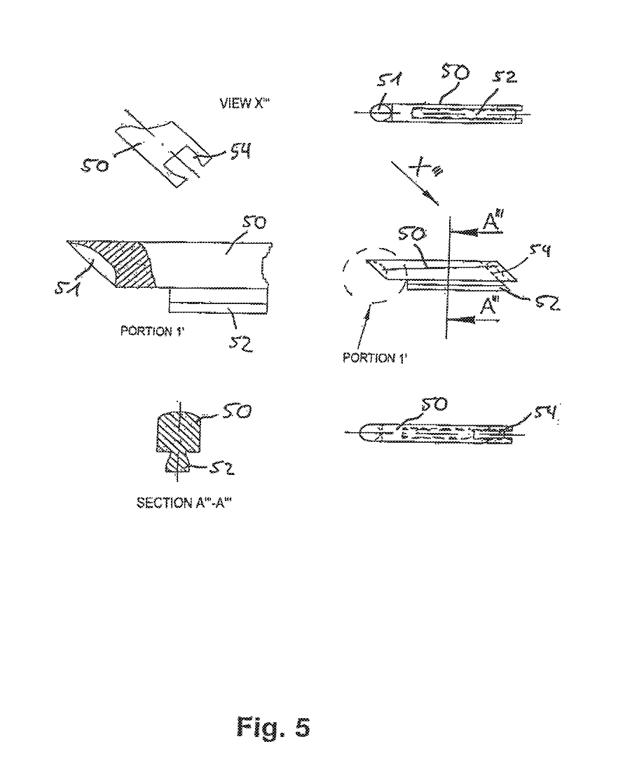

FIG. 5 in detail shows several views and details of the second punching means 50. In its part facing the movable part 30, the second punching means 50 includes a dovetail groove 54 in which the dovetail tongue 34 of the movable part 30 engages. Furthermore, at the bottom surface of the punching means 50 a dovetail tongue 52 or a guiding element 52 is provided, which can engage in the dovetail groove 24 of the main part 20. The second punching means 50 preferably is formed of hardened surgical steel with a hardness of 56-60 HRC.

1. A surgical instrument comprising a main part with a first punching means and a part movable relative thereto, with a handle arranged on the main part, which includes a stationary grip and a grip which can actuate the movable part, wherein the movable part is releasably connected with a second punching means, wherein the second punching means is joinable with the first punching means for a punching operation and is movably guidable or guided on the main part along with the movable part.

2. The surgical instrument according to claim 1, wherein both the second punching means and the movable part include guiding means, by means of which the second punching means and the movable part are movably guidable or guided in a guideway in the main part.

3. The surgical instrument according to claim 2, wherein the second punching means and the movable part are positively movably guidable or guided on or in the main part.

4. The surgical instrument according to claim 2, wherein the main part includes an at least partly formed groove in which the second punching means and the movable part are at least partly movably guidable or guided on or in the main part.

5. The surgical instrument according to claim 4, wherein the groove is dovetailed.

6. The surgical instrument according to claim 5, wherein the guiding means of the second punching means and of the movable part are at least partly dovetailed and at least partly positively engage in the groove.

7. The surgical instrument according to claim 1, wherein the movable part is positively releasably connected with the second punching means.

8. The surgical instrument according to claim 7, wherein the positively releasable connection between the movable part and the second punching means is at least partly dovetailed.

9. The surgical instrument according to claim 1, wherein the second punching means is formed of a hardened material.

10. The surgical instrument according to claim 9, wherein the hardness of the second punching means and/or of the first punching means is at least 45 HRC.

11. The surgical instrument according to claim 9, wherein the surgical instrument is dismantleable into all its parts.

12. The surgical instrument according to claim 11, wherein at least one unlocking means is present, by means of which the main part and the movable part can be unlocked or detached from the handle.

13. A surgical instrument comprising:
a main part with a handle arranged thereon including a stationary first grip and a movable second grip, the main part including a first punch;
a part movable relative to the main part, wherein the second grip is coupled to the movable part to actuate the movable part; and
a second punch, wherein the movable part is releasably connected with the second punch, and the second punch is joinable with the first punch during a punching operation, and wherein the first punch is guided by the main part along with the movable part.

14. The surgical instrument according to claim 13, wherein the first punch is integrally formed at the main part as an L-shaped upstanding formation and includes a tray-like recess having sharp-edged ends, and wherein the second punch is shaped as a counterpart to the first punch such that tissue material can be cut off therewith.

15. The surgical instrument according to claim 14, wherein both the second punch and the movable part include a guide element by which the second punch and the movable part are guided, and wherein the main part includes a partly formed groove in which the second punch and the movable part are movably guided on or in the main part.

16. The surgical instrument according to claim 15, wherein the groove is dovetailed.

17. The surgical instrument according to claim 16, wherein the guide is dovetailed and is positively engaged in the groove.

18. The surgical instrument according to claim 17, wherein the second punch comprises a hardened surgical steel.

19. The surgical instrument according to claim 19, further comprising an unlocking pin, by which the main part and the movable are unlocked from one another and detachable from the handle.

20. A surgical instrument comprising:
an elongate main part with a handle arranged thereon including a stationary first grip and a movable second grip, the main part including a first elongate punch, the handle sized shaped to be squeezed by a human hand;
a part movable relative to the main part, wherein the second grip is coupled to the movable part to actuate the movable part; and
a second punch, wherein the movable part is releasably connected with the second punch, and the second punch is joinable with the first punch during a punching operation, and wherein the first punch is guided by the main part along with the movable part.
